(19) United States
(12) Patent Application Publication
(10) Pub. No.: US 2023/0348546 A1
(43) Pub. Date: Nov. 2, 2023

(54) METHOD FOR BIOSYNTHESIS OF PROTEIN HETEROCATENANE

(71) Applicant: Peking University, Beijing (CN)

(72) Inventors: Wenbin ZHANG, Beijing (CN); Yajie LIU, Beijing (CN)

(21) Appl. No.: 17/999,377

(22) PCT Filed: May 19, 2021
(86) PCT No.: PCT/CN2021/094589

§ 371 Date: Nov. 18, 2022

(30) Foreign Application Priority Data
May 21, 2020 (CN) 202010436910.X

(51) Int. Cl. C07K 14/47; C12P 21/02; C12N 15/70

(52) U.S. Cl. CPC C07K 14/4746; C12P 21/02; C12N 15/70; C07K 2319/92; C07K 2319/21; C07K 2319/50

(57) ABSTRACT

Provided is a method for biosynthesis of a protein heterocatenane. The basic structure of a protein precursor sequence of the protein heterocatenane comprises form an N-terminal to a C-terminal: L₁₋₁-X-L₁₋₂-(in situ enzyme cutting site)-L₂₋₁-X-L₂₋₂, wherein the Xs represent entangled motifs for forming dimers, the two Xs can be the same or different, L₁₋₁/L₁₋₂ and L₂₋₁/L₂₋₂ represent two pairs of cyclization motifs that undergo an orthogonal coupling reaction in cellulo, and the two pairs of cyclization motifs can be two orthogonal peptide-protein reactive pairs, or combinations of peptide-protein reactive pairs and split inteins, or two orthogonal split inteins. When the peptide-protein reactive pair and the split intein are combined for use, biosynthesis of branched protein heterocatenanes can be achieved; and when the two orthogonal split inteins are combined for use, the protein heterocatenane having a completely cyclized main chain can be obtained.

### CROSS-REFERENCE TO RELATED APPLICATION

The present application claims the benefit of a priority of Chinese Patent Application No. 202010436910.X, entitled “METHOD FOR BIOSYNTHESIS OF PROTEIN HETEROGENEOUS CATENANE” and filed on May 21, 2020, the entire contents of which including the Appendix are incorporated herein by reference.

### TECHNICAL FIELD

The present disclosure relates to a method for biosynthesis of a protein heterocatenane, in particular to a biosynthetic system based on peptide-protein reactive pairs and/or split inteins, and a method for constructing a multi-domain protein heterocatenane by two orthogonal coupling cyclization modes on the basis of said system.

### BACKGROUND

In nature, many natural biological macromolecules have specific topology and are closely related to their respective biological functions. Natural topological proteins that have so far been found include cyclic proteins, knotted proteins, lasso proteins, and protein catenanes, etc. Since the construction of cyclic proteins requires only the coupling of polypeptide chains, it is currently the focus of artificial topological proteins, which typically shows significantly improved thermal stability. Due to the complexity of the mechanism of protein folding, it is relatively difficult to regulate the topology of proteins by controlling the entwining relationship between polypeptide chains. The simplest [2]catenane among catenanes is composed of two mechanically interlocked cyclic motifs, and hence the corresponding protein heterocatenane structure can not only possess the advantages of cyclic proteins, but also achieve synergy functions by regulating the relative positions of the two cyclic motifs. Nevertheless, such a structure has not been found in nature. It is thus a very attractive research direction to develop a preparation method for protein heterocatenanes.

Relatively few reports are currently available on the synthesis of artificial protein catenanes, and their synthetic strategies can be broadly classified into three categories while the essence of achieving mechanically interlocked structures is all based on the folded structure of the proteins. The first category is realizing synthesis of protein homocatenanes by guiding the intertwining among molecular chains using the tetrameric domain p53tet of the tumor suppressor protein p53 or its mutant dimeric domain p53dim, followed by cyclization through highly efficient specific natural chemical ligation or SpyTag-SpyCatcher reactive pairs. The second category is gradually converting the topology of lasso peptides into higher-order catenanes through enzyme digestion and assembly. In the third category, the synthesis of protein heterocatenanes was achieved for the first time by splitting SpyCatcher into BDTag and SpyStapler and rationally recombining the three motifs based on the folded structure of the SpyTag-SpyCatcher reactive pairs combined with the characteristics of the split intein-mediated cyclization and autocatalytic formation of isopeptide bonds, but the reaction cannot be complete and the whole purification process is tedious. Based on the assembly-reaction synergy, further development of methods for biosynthesis of protein heterocatenanes will contribute to a more in-depth study on the effects of the topology on the protein functions and properties and will also lay the foundation for their applications in the field of biomedicine.

### SUMMARY

An objective of the present disclosure is to provide a strategy for biosynthesis of protein heterocatenanes that allows for efficient construction of multi-domain protein heterocatenanes without implementing any additional extracellular reactions.

By mimicking the multi-step post-translational modification process in the synthesis of natural topological proteins, combined with the in situ assembly, chain cleavage and site-specific cyclization, the present disclosure develops a synthetic system based on two orthogonal coupling approaches through rationally designed gene sequences, which enables modular synthesis of protein heterocatenanes featuring a branched or fully backbone cyclized structure.

The basic structure of the protein precursor sequence designed herein for preparing the protein heterocatenane includes: L₁₋₁-X-L₁₋₂-(in situ enzyme digestion site)-L₂₋₁-X-L₂₋₂, wherein
(1) X represents an entwining motif that can form a dimer and is one of the key elements for the formation of heterocatenanes; two Xs may be the same, e.g., homodimer-forming entwining motifs such as a tumor suppressor-derived p53dim domain or a HP0242 protein from Helicobacter pylori; the two Xs may also be different, e.g., heterodimer motifs derived by substitution of amino acid residues or the like on the basis of the above dimer motifs, or native entwined heterodimeric motifs in nature; and
(2) L₁₋₁/L₁₋₂ and L₂₋₁/L₂₋₂ represent two pairs of cyclization motifs that undergo orthogonal coupling reactions in cellulo and are another key element for the formation of heterocatenanes. The cyclization motif may be selected from peptide-protein reactive pair, a split intein and the like; in order to avoid excessive side reactions, the two cyclization approaches should be somewhat orthogonal. In order to realize the synthesis of heterocatenanes, in specific cases it may be desirable to insert an in situ enzyme digestion site between L₁₋₂ and L₂₋₁, which can be cleaved in situ by intracellularly co-expressed protease, for example by inserting the recognition site of the TVMV protease.

The two pairs of cyclization motifs are selected mainly from the following three options:
(1) Two orthogonal peptide-protein reactive pairs, such as a SpyTag-SpyCatcher reactive pair and a SnoopTag-SnoopCatcher reactive pair. Under this circumstance, an in situ enzyme digestion site must be inserted between the two reactive pairs to convert one polypeptide chain into two polypeptide chains by co-expressing the protease.
(2) Combinations of a peptide-protein reactive pair and a split intein, such as a SpyTag-SpyCatcher reactive pair and an NpuDnaE split intein (including the C-terminal part and the N-terminal part). When the polypeptide-protein reactive pair is ahead of the split intein, i.e., L₁₋₁/L₁₋₂ is a polypeptide-protein reactive pair and L₂₋₁/L₂₋₂ is a split intein, since the intracellular cyclization reaction of the polypeptide-protein reactive pair is a side-chain coupling reaction and the resulting complex will exist in the final structure, there is a need to initiate the cyclization reaction of L₂₋₁/L₂₋₂ by the in situ enzyme digestion; when the split intein is ahead of the polypeptide-protein reactive pair, i.e., L₁₋₁/L₁₋₂ is a split intein and L₂₋₁/L₂₋₂ is a polypeptide-protein reactive pair, due to the characteristics that the split intein-mediated cyclization is backbone coupling and after cyclization, the split intein will be released from the precursor protein by means of self-splicing, the in situ enzyme digestion may not be necessary.
(3) Two orthogonal split inteins, such as IntC1/IntN1 and IntC2/IntN2 formed by splitting NpuDnaE intein in two different ways and other split inteins such as gp41-1, gp41-8, NrdJ-1, and IMPDH-1. Two split inteins may be used as long as they are somewhat orthogonal. The split intein-mediated cyclization has the advantages of forming backbone cyclization and removing split inteins by self-splicing with few redundant amino acids left. In case of using two orthogonal split inteins, an in situ enzyme digestion site may not be inserted.

By inserting one or more identical or different proteins of interest in the basic structure of the above-mentioned protein precursor sequence, it is possible to construct a protein heterocatenane comprising the proteins of interest. The insertion sites for the proteins of interest may be within the ring, i.e., before and/or after the X domain. Since the cyclization mediated by the peptide-protein reactive pair is side-chain coupling and the N terminus and C terminus are still intact after cyclization, the insertion sites for the proteins of interest may also be outside the ring. i.e., the N terminus and/or the C terminus of the peptide-protein reactive pair, thereby constructing a branched heterocatenane.

The gene construction of the target protein is shown in FIG. 1. In the protein precursor sequence L₁₋₁-X-POI1-L₁₋₂-(TVMV)-L₂₋₁-X-POI2-L₂₋₂, L₁₋₁/L₁₋₂ and L₂₋₁/L₂₋₂ represent two cyclization motifs in orthogonal cyclization manner, X represents an entwining motif, POI1 and POI2 represent protein 1 of interest and protein 2 of interest; the TVMV site represents the recognition site of the TVMV protease, which can be recognized and in situ digested by the co-expressed TVMV protease; a purification tag (such as a histidine tag) is introduced before the second entwining motif X to facilitate purification of the synthesized heterocatenane. The positions at which the proteins of interest may be fused are enumerated as follows:
(1) When L₁₋₁/L₁₋₂ and L₂₋₁/L₂₋₂ are orthogonal peptide-protein reactive pairs, both of them undergo side-chain coupling cyclization, the in situ enzyme digestion is needed, and the resulting complexes L₁ and L₂ will exist in the final catenane structure. Therefore, in addition to formation of a heterocatenane cat-L₁(X-POI1)-L₂(X-POI2) by inserting the proteins of interest POI1 and POI2 into two rings respectively, a branched heterocatenane may be constructed by further fusing the proteins of interest (POI3, POI4, POI5, POI6) at the N termini and C termini of the peptide-protein reactive pairs, and the positions where the proteins of interest are inserted are as follows: POI3-L₁₋₁-X-POI1-L₁₋₂-POI4-(TVMV)-POI5-L₂₋₁-X-POI2-L₂₋₂-POI6.
(2) When L₁₋₁/L₁₋₂ and L₂₋₁/L₂₋₂ are combinations of a peptide-protein reactive pair and a split intein, since the complex formed by the split intein would be removed by self-splicing, if L₁₋₁/L₁₋₂ is a peptide-protein reactive pair and L₂₋₁/L₂₋₂ is a split intein, the heterocatenane formed by inserting the proteins of interest POI1 and POI2 into two rings respectively is cat-L₁(X-POI1)-(X-POI2). A branched heterocatenane may be constructed by further fusing the proteins of interest (POI3, POI4) at the N terminus and C terminus of the L₁₋₁/L₁₋₂ peptide-protein reactive pair, and the positions of POI insertion are as follows: POI3-L₁₋₁-X-POI1-L₁₋₂-POI4-(TVMV)-L₂₋₁-X-POI2-L₂₋₂. Otherwise, if L₁₋₁/L₁₋₂ is a split intein and L₂₋₁/L₂₋₂ is a peptide-protein reactive pair, a heterocatenane cat-(X-POI1)-L₂(X-POI2) will be formed. A branched heterocatenane may be constructed by further fusing the proteins of interest (POI3, POI4) at the N terminus and C terminus of the L₂₋₁/L₂₋₂ peptide-protein reactive pair, and the positions of POI insertion are as follows: L₁₋₁-X-POI1-L₁₋₂-(TVMV)-POI3-L₂₋₁-X-POI2-L₂₋₂-POI4.
(3) When L₁₋₁/L₁₋₂ and L₂₋₁/L₂₋₂ are orthogonal split inteins, since the complex formed by the split inteins would be removed by self-splicing which mediates the backbone cyclization, the heterocatenane formed by inserting the proteins of interest POI1 and POI2 into two rings respectively is cat-(X-POI1)-(X-POI2), in which both of the two cyclic protein moieties are backbone-cyclized and no other redundant components than the entwining motifs and proteins of interest are included.

The strategy adopted in the present disclosure for biosynthesis of protein heterocatenanes focuses on the following aspects: (1) entwining motifs (X) such as the p53dim domain are utilized to realize mechanical interlocking, and the yield of heterocatenanes is improved by converting intermolecular dimerization into intramolecular dimerization; (2) cyclization modes that can occur intracellularly are selected, and peptide-protein reactive pairs and split inteins are most widely used at present; (3) the two cyclization modes should be somewhat orthogonal to avoid excessive side reactions, for example, a SpyTag-SpyCatcher reactive pair is used in combination with a split intein, or two split inteins with certain orthogonality are selected; and (4) a split intein typically includes a large-sized N-terminal part (IntN) and a relatively small-sized C-terminal part (IntC), and when IntC is located in the chain resulting in a blocked reaction, in situ cleavage of the nascent polypeptide chain by co-expressing the protease can trigger the trans-splicing reaction mediated by this split intein.

The split intein involved herein is preferably an NpuDnaE split intein, which is naturally split into IntC1 containing 36 amino acids and IntN1 containing 102 amino acids. IntC2 containing 15 amino acids and the corresponding IntN2 containing 123 amino acids, obtained by systematically truncating the IntC part, also have a good trans-splicing efficiency. Although IntC1 is somewhat reactive with IntN2, IntC2 is unable to react with IntN1, reflecting certain orthogonality.

The biosynthetic systems for the protein heterocatenanes described herein all make use of the intramolecular dimerization of entwining motifs such as the p53dim domain to guide the entwining of the polypeptide chains, but achieve orthogonal coupling in different ways. The intracellular cyclization reaction based on peptide-protein reactive pairs is a side-chain coupling reaction with intact N-/C-termini, while the resulting complex exists in the final structure, and thus a branched protein heterocatenane can be prepared by further fusing other proteins of interest. In contrast, the intracellular cyclization reaction based on the split inteins can realize the backbone cyclization by linking the two ends of the peptide chain via a native peptide bond, while the split inteins are released from the precursor proteins by self-splicing.

The method for biosynthesis of a protein heterocatenane provided herein substantially comprises:
1) designing a protein precursor sequence of the protein heterocatenane with a basic structure including, from the N terminus to the C terminus: L₁₋₁-X-L₁₋₂-(in situ enzyme digestion site)-L₂₋₁-X-L₂₋₂, wherein X represents a dimer-forming entwining motif; L₁₋₁/L₁₋₂ and L₂₋₁/L₂₋₂ represent two pairs of cyclization motifs that undergo an orthogonal coupling reaction in cellulo, which can be two orthogonal peptide-protein reactive pairs, or combinations of a peptide-protein reactive pair and a split intein, or two orthogonal split inteins; when L₁₋₁/L₁₋₂ is the peptide-protein reactive pair, the in situ protease digestion site inserted between L₁₋₂ and L₂₋₁ is an essential element, which can be digested in situ by co-expressing a protease intracellularly; otherwise the in situ protease digestion site is a non-essential element; the sequence of a protein of interest is inserted in the above basic structure, and the insertion sites are selected from: before and/or after the X domain, at the N terminus and/or at the C terminus of the peptide-protein reactive pair;
2) constructing the gene sequence encoding the protein precursor sequence described in step 1) and introducing the gene sequence into an expression vector;
3) transforming the expression vector constructed in step 2) into a cell for expression, and co-expressing, if necessary, the protease that in situ cleaves the digestion site in cellulo; and
4) purifying a fusion protein obtained in step 3) to give the corresponding protein heterocatenane.

In step 1) described above, the peptide-protein reactive pair is preferably a SpyTag-SpyCatcher reactive pair or a SnoopTag-SnoopCatcher reactive pair. The amino acid sequences of typical SpyTag and SpyCatcher are as shown in SEQ ID NO:1 and SEQ ID NO:2 in the sequence listing. A reactive SpyTag/SpyCatcher mutant may also be used. The mutant refers to a peptide chain derived from the above amino acid sequence of SpyTag/SpyCatcher by substitution, deletion or addition of amino acid residue(s), where the substitution, deletion or addition of amino acid residue(s) does not exert any influence on the coupling reaction for generating isopeptide bonds.

In step 1) described above, the entwining motif X is preferably a tumor suppressor-derived p53dim domain. The amino acid sequence of typical p53dim domain is as shown in SEQ ID NO:3 in the sequence listing. A p53dim mutant capable of forming an analogous dimeric structure may be used. The mutant refers to a peptide chain derived from the above amino acid sequence by substitution, deletion or addition of amino acid residue(s), where the substitution, deletion or addition of amino acid residue(s) does not exert any influence on the generation of their entwined dimers.

In step 1) described above, the split intein is preferably an NpuDnaE split intein containing N-terminal part (IntN) and C-terminal part (IntC) to constitute a cyclization motif, and the amino acid sequences of IntC1, IntN1, IntC2 and IntN2 resulting from the two splitting methods are as shown in SEQ ID NO:4, SEQ ID NO:5, SEQ ID NO:6 and SEQ ID NO:7 in the sequence listing. In addition, other eligible split inteins may also be applied in the present disclosure to enable the biosynthesis of protein heterocatenanes.

In step 1) described above, the in situ enzyme digestion site is preferably a recognition sequence ETVRFQG of the Tobacco vein mottling virus (TVMV) protease.

In step 1) described above, in order to demonstrate the topology of the synthesized protein heterocatenane, a recognition sequence ENLYFQG of the Tobacco etch virus (TEV) protease may be introduced before the first entwining motif X, which may also be used as the in situ enzyme digestion site as described. To facilitate purification, a histidine tag is further introduced before the second entwining motif X. The protein purification is performed by affinity chromatography on a nickel column in step 4).

In step 3) described above, in the case of L₁₋₁/L₁₋₂ being a peptide-protein reactive pair, co-expression with a protease to digest the cleavage site in situ is necessary to achieve the biosynthesis of protein heterocatenanes; in the case of L₁₋₁/L₁₋₂ being a split intein, there is no need to co-express the protease.

In step 4) described above, for the protein heterocatenane with a histidine tag introduced therein, the expressed protein is purified by affinity chromatography on a nickel column, and the purity of the protein heterocatenane can be further improved by gradient elution or size exclusion chromatography.

In the examples of the present disclosure, the following protein precursor sequences are designed as shown in FIG. 2:
SpyCatcher(B)-p53dim(X)-SpyTag(A)-IntC1-p53dim(X)-IntN1, abbreviated as BXA-IntC1-X-IntN1;
IntC1-p53dim(X)-POI1-IntN1-IntC2-p53dim(X)-POI2-IntN2, abbreviated as IntC1-X-POI1-IntN1-IntC2-X-POI2-IntN2.

The above coding genes are introduced into the expression vector pMCSG19, which is then transformed into BL21(DE3) competent cells for expression. In the system BXA-IntC1-X-IntN1 where the protease needs to be co-expressed. BL21(DE3) competent cells also contain a pRK1037 plasmid encoding the TVMV protease; in contrast, IntC1-X-POI1-IntN1-IntC2-X-POI2-IntN2 enables the biosynthesis of protein heterocatenanes when expressed alone or co-expressed with the TVMV protease, and there is no significant difference between the two conditions, so transferring the expression vectors into conventional BL21(DE3) competent cells for expression is sufficient. Finally, the obtained fusion proteins are purified to obtain the corresponding protein heterocatenanes.

A protein precursor obtained by expressing a recombinant plasmid of BXA-IntC1-X-IntN1 or IntC1-X-POI1-IntN1-IntC2-X-POI2-IntN2 first forms an intramolecularly entwined structure by dimerization of the p53dim domain and then achieves site-specific cyclization by two orthogonal coupling approaches. In the BXA-IntC1-X-IntN1 system, it is necessary to achieve the in situ enzyme digestion by co-expression with the TVMV protease to trigger the IntC1/IntN1-mediated trans-splicing reaction, followed by the side-chain cyclization reaction mediated by the SpyTag-SpyCatcher reactive pairs, resulting in the preparation of protein heterocatenane cat-BXA-X. In the system of IntC1-X-POI1-IntN1-IntC2-X-POI2-IntN2, two pairs of split inteins can undergo the sequential trans-splicing reaction to mediate the cyclization of the two proteins of interest in turn, ultimately leading to the preparation of protein heterocatenane cat-XPOI1-XPOI2.

By introducing other folded proteins, such as AffiHER2 with high affinity for HER2, at the N terminus of SpyCatcher and the C terminus of SpyTag on the basis of BXA-IntC1-X-IntN1, it is possible to realize biosynthesis of branched protein heterocatenanes based on the same co-expression method. In the system of IntC1-X-POI1-IntN1-IntC2-X-POI2-IntN2, a small ubiquitin-modified protein SUMO and a superfolded protein GFP are selected as model proteins to achieve the biosynthesis of protein heterocatenanes cat-XSUMO-X and cat-XSUMO-XGFP, respectively.

### DETAILED DESCRIPTION OF THE INVENTION

Before describing the present disclosure in detail, it should be appreciated that the present disclosure is not subject to the particular methods and experimental conditions described herein because the methods and conditions can be altered. Furthermore, the terms used herein are only for the purpose of explaining particular embodiments and are not intended to be limiting.

Unless otherwise defined, all technical and scientific terms used herein have the same meanings as conventionally understood by a person skilled in the art. For the sake of the present disclosure, the following terms are defined.

The term “and/or”, when used to connect two or more options, shall be understood to mean either of or any two or more of the options.

As used herein, the term “comprising” or “including” is intended to include the elements, integers or steps, without the exclusion of any other elements, integers or steps. The term “comprising” or “including”, when used herein, also covers situations of consisting of the recited elements, integers or steps, unless otherwise indicated.

Various exemplary examples, features, and aspects of the present disclosure will be illustrated in detail below. The term “exemplary” as used exclusively herein means “serving as an instance, example or illustration”. Any example described herein as “exemplary” is not necessarily construed as superior to or better than the other examples.

In addition, numerous details are set forth in the specific embodiments below in order to better illustrate the present disclosure. It shall be appreciated to a person skilled in the art that the present disclosure can still be implemented even without some of the details. In some other examples, methods, means, equipment, and steps familiar to a person skilled in the art are not described in detail so as to highlight the principles of the present disclosure.

Unless otherwise stated, all of the units used in the present specification are international standard units and all of the numerical values and numerical ranges used herein shall be construed as inclusion of systematic errors unavoidable in industrial production.

The sequences of protein precursors involved in the biosynthesis of protein heterocatenanes are illustrated below by way of some specific examples:
(a) SpyCatcher(B)-p53dim(X)-SpyTag(A)-IntC1-p53dim(X)-IntN1(BXA-IntC1-X-IntN1): from the N terminus to the C terminus are a reaction motif SpyCatcher, an entwining motif p53dim domain, a reaction motif SpyTag, a C-terminal part IntC1 of the split intein, an entwining motif p53dim domain, and an N-terminal part IntN1 of the split intein, respectively. In the sequence, a recognition sequence of the TEV protease is inserted between the SpyCatcher and the first p53dim domain, a recognition sequence of the TVMV protease is inserted between the SpyTag and the IntC1, and a histidine tag is introduced before the second p53dim domain. The gene sequence of BXA-IntC1-X-IntN1 is as shown in SEQ ID No:8 in the sequence listing, in which the amino acid residues 8-122 are SpyCatcher, the amino acid residues 132-138 are the recognition sequence of the TEV protease, the amino acid residues 186-198 are SpyTag, the amino acid residues 143-180 and 274-311 are the p53dim domain, the amino acid residues 205-211 are the recognition sequence of the TVMV protease, the amino acid residues 221-255 are IntC1, the amino acid residues 261-266 are 6×His tag, and the amino acid residues 319-420 are IntN1.
(b) AffiHER2-SpyCatcher(B)-p53dim(X)-SpyTag(A)-AffiHER2-IntC1-p53dim(X)-IntN1 (AffiHER2-BXA-AffiHER2-IntC1-X-IntN1): from the N terminus to the C terminus are a protein of interest AffiHER2, a reaction motif SpyCatcher, an entwining motif p53dim domain, a reaction motif SpyTag, a protein of interest AffiHER2, a C-terminal part IntC1 of the split intein, an entwining motif p53dim domain, and an N-terminal part IntN1 of the split intein, respectively. In the sequence, a recognition sequence of the TEV protease is inserted between the SpyCatcher and the first p53dim domain, a recognition sequence of the TVMV protease is inserted between the second AffiHER2 and IntC1, and a histidine tag is introduced before the second p53dim domain. The gene sequence of AffiHER2-BXA-AffiHER2-IntC1-X-IntN1 is as shown in SEQ ID No:9 in the sequence listing, in which the amino acid residues 6-75 and 279-348 are AffiHER2, the amino acid residues 82-196 are SpyCatcher, the amino acid residues 206-212 are the recognition sequence of the TEV protease, the amino acid residues 260-272 are SpyTag, the amino acid residues 217-254 and 424-461 are the p53dim domain, the amino acid residues 355-361 are the recognition sequence of the TVMV protease, the amino acid residues 371-405 are IntC1, the amino acid residues 411416 are 6-His tag, and the amino acid residues 469-570 are IntN1.
(c) IntC1-p53dim(X)-SUMO-IntN1-IntC2-p53dim(X)-IntN2 (IntC1-X-SUMO-IntN1-IntC2-X-IntN2): from the N terminus to the C terminus are a C-terminal part IntC1 of the split intein, an entwining motif p53dim domain, a protein of interest SUMO, an N-terminal part IntN1 of the split intein, a C-terminal part IntC2 of the split intein, an entwining motif p53dim domain, and an N-terminal part IntN2 of the split intein, respectively. In the sequence, a recognition sequence of the TEV protease is inserted between the IntC1 and the first p53dim domain, a recognition sequence of the TVMV protease is inserted between IntN1 and IntC2, and a histidine tag is introduced before the second p53dim domain. The gene sequence of IntC1-X-SUMO-IntN1-IntC2-X-IntN2 is as shown in SEQ ID No:10 in the sequence listing, in which the amino acid residues 8-42 are IntC1, the amino acid residues 48-54 are the recognition sequence of the TEV protease, the amino acid residues 62-99 and 358-395 are the p53dim domain, the amino acid residues 100-195 are the protein of interest SUMO, the amino acid residues 203-304 are IntN1, the amino acid residues 311-317 are the recognition sequence of the TVMV protease, the amino acid residues 345-350 are 6-His tag, the amino acid residues 326-339 are IntC2, and the amino acid residues 403-504 are IntN2.
(d) IntC1-p53dim(X)-SUMO-IntN1-IntC2-p53dim(X)-GFP-IntN2 (IntC1-X-SUMO-IntN1-IntC2-X-GFP-IntN2): from the N terminus to the C terminus are a C-terminal part IntC1 of the split intein, an entwining motif p53dim domain, a protein of interest SUMO, an N-terminal part IntN1 of the split intein, a C-terminal part IntC2 of the split intein, an entwining motif p53dim domain, a protein of interest GFP, and an N-terminal part IntN2 of the split intein, respectively. In the sequence, a recognition sequence of the TEV protease is inserted between IntC1 and the first p53dim domain, a recognition sequence of the TVMV protease is inserted between IntN1 and IntC2, and a histidine tag is introduced before the second p53dim domain. The gene sequence of IntC1-X-SUMO-IntN1-IntC2-X-GFP-IntN2 is as shown in SEQ ID No:11 in the sequence listing, in which the amino acid residues 8-42 are IntC1, the amino acid residues 48-54 are the recognition sequence of the TEV protease, the amino acid residues 62-99 and 358-395 are the p53dim domain, the amino acid residues 100-195 are the protein of interest SUMO, the amino acid residues 203-304 are IntN1, the amino acid residues 311-317 are the recognition sequence of the TVMV protease, the amino acid residues 345-350 are 6×His tag, the amino acid residues 326-339 are IntC2, the amino acid residues 403-640 are the protein of interest GFP, and the amino acid residues 643-765 are IntN2.

The present disclosure carries out the basic characterization and topological proof of the prepared protein heterocatenanes by conventional characterization means such as sodium dodecyl sulfate polyacrylamide gel electrophoresis (SDS-PAGE), ultra-performance liquid chromatography-mass spectrometry (LC-MS) and TEV protease digestion reaction.

Based on a rational design of gene sequences to combine in situ assembly, enzyme digestion and site-specific cyclization, the present disclosure develops an orthogonal coupling-based biosynthetic system applicable to the intracellular synthesis of heterocatenanes containing a variety of functional proteins, which highlights the following major advantages: 1) said biosynthetic system enables modular synthesis of heterocatenanes by genetically encoded approach, and improves the yield of protein heterocatenanes using intramolecular dimerization of entwining dimeric motifs such as the p53dim domain, and there are a variety of options available for the corresponding entwining motifs and coupling means; 2) by mimicking the multi-step post-translational modification process in synthesis of natural topological proteins, said biosynthetic system accomplishes the entwining of polypeptide chains and two orthogonal covalent cyclization reactions intracellularly without the need for additional extracellular reactions, and the corresponding protein heterocatenanes are obtained after expression and purification; and 3) in the construction of a protein precursor containing a peptide-protein reactive pair such as BXA-IntC1-X-IntN1, biosynthesis of branched protein heterocatenanes can be realized by introducing other folded proteins at the N terminus of the SpyCatcher and the C terminus of the SpyTag; while in the construction of a protein precursor containing two orthogonal split inteins such as IntC1-X-POI1-IntN1-IntC2-X-POI2-IntN2, biosynthesis of protein heterocatenanes with backbone cyclization is achieved. Both systems extend the scope of the existing protein heterocatenane structures.

| | |
|---|---|
| SEQ ID No: 1 | AHIVMVDAYKPTK |
| SEQ ID No: 2 | AMVDTLSGLSSEQGQSGDMTIEEDSATHIK |
| | FSKRDEDGKELAGATMELRDSSGKTISTWI |
| | SDGQVKDFYLYPGKYTFVETAAPDGYEVAT |
| | AITFTVNEQGQVTVNGKATKGDAHI |
| SEQ ID No: 3 | GGEYFTLQIRGRERFEEFREKNEALELKDA |
| | QAGKEPGG |
| SEQ ID No: 4 | IKIATRKYLGKQNVYDIGVERDHNFALKNG |
| | FIASN |
| SEQ ID No: 5 | CLSYETEILTVEYGLLPIGKIVEKRIECTV |
| | YSVDNNGNIYTQPVAQWHDRGEQEVFEYCL |
| | EDGSLIRATKDHKFMTVDGQMLPIDEIFER |
| | ELDLMRVDNLPN |
| SEQ ID No: 6 | DHNFALKNGFIASN |
| SEQ ID No: 7 | CLSYETEILTVEYGLLPIGKIVEKRIECTV |
| | YSVDNNGNIYTQPVAQWHDRGEQEVFEYCL |
| | EDGSLIRATKDHKFMTVDGQMLPIDEIFER |
| | ELDLMRVDNLPNIKIATRKYLGKQNVYDIG |
| | VER |
| SEQ ID No: 8 | MKGSSASAMVDTLSGLSSEQGQSGDMTIEE |
| | DSATHIKFSKRDEDGKELAGATMELRDSSG |
| | KTISTWISDGQVKDFYLYPGKYTFVETAAP |
| | DGYEVATAITFTVNEQGQVTVNGKATKGDA |
| | HIDGPQGIWGQENLYFQGGSGSGGEYFTLQ |
| | IRGRERFEEFREKNEALELKDAQAGKEPGG |
| | SGGSGAHIVMVDAYKPTKVDSGSGETVRFQ |
| | GGGSGGSSGMIKIATRKYLGKQNVYDIGVE |
| | RDHNFALKNGFIASNCFNGGHHHHHHELSG |
| | SGSGGEYFTLQIRGRERFEEFREKNEALEL |
| | KDAQAGKEPGGSGGSGTSCLSYETEILTVE |
| | YGLLPIGKIVEKRIECTVYSVDNNGNIYTQ |
| | PVAQWHDRGEQEVFEYCLEDGSLIRATKDH |
| | KFMTVDGQMLPIDEIFERELDLMRVDNLPN |
| SEQ ID No: 9 | MKGSSTGGQQMGRDPGVDNKFNKEMRNAYW |
| | EIALLPNLNNQQKRAFIRSLYDDPSQSANL |
| | LAEAKKLNDAQAPKGGGGSASAMVDTLSGL |
| | SSEQGQSGDMTIEEDSATHIKFSKRDEDGK |
| | ELAGATMELRDSSGKTISTWISDGQVKDFY |
| | LYPGKYTFVETAAPDGYEVATAITFTVNEQ |
| | GQVTVNGKATKGDAHIDGPQGIWGQENLYF |
| | QGGSGSGGEYFTLQIRGRERFEEFREKNEA |
| | LELKDAQAGKEPGGSGGSGAHIVMVDAYKP |
| | TKGTGGSMTGGQQMGRDPGVDNKFNKEMRN |
| | AYWEIALLPNLNNQQKRAFIRSLYDDPSQS |
| | ANLLAEAKKLNDAQAPKGVDSGSGETVRFQ |
| | GGGSGGSSGMIKIATRKYLGKQNVYDIGVE |
| | RDHNFALKNGFIASNCFNGGHHHHHHELSG |
| | SGSGGEYFTLQIRGRERFEEFREKNEALEL |
| | KDAQAGKEPGGSGGSGTSCLSYETEILTVE |
| | YGLLPIGKIVEKRIECTVYSVDNNGNIYTQ |
| | PVAQWHDRGEQEVFEYCLEDGSLIRATKDH |
| | KFMTVDGQMLPIDEIFERELDLMRVDNLPN |
| SEQ ID No: 10 | MKGSSASIKIATRKYLGKQNVYDIGVERDH |
| | NFALKNGFIASNCFNGGENLYFQGRSSGSG |
| | SGGEYFTLQIRGRERFEEFREKNEALELKD |
| | AQAGKEPGGDSEVNQEAKPEVKPEVKPETH |
| | INLKVSDGSSEIFFKIKKTTPLRRLMEAFA |
| | KRQGKEMDSLRFLYDGIRIQADQTPEDLDM |
| | EDNDIIEAHREQIGGSGGSGGTCLSYETEI |
| | LTVEYGLLPIGKIVEKRIECTVYSVDNNGN |
| | IYTQPVAQWHDRGEQEVFEYCLEDGSLIRA |
| | TKDHKFMTVDGQMLPIDEIFERELDLMRVD |
| | NLPNVDSGSGETVRFQGGGSGGSSGDHNFA |
| | LKNGFIASNCFNGGHHHHHHELSGSGSGGE |
| | YFTLQIRGRERFEEFREKNEALELKDAQAG |
| | KEPGGSGGSGTSCLSYETEILTVEYGLLPI |
| | GKIVEKRIECTVYSVDNNGNIYTQPVAQWH |
| | DRGEQEVFEYCLEDGSLIRATKDHKFMTVD |
| | GQMLPIDEIFERELDLMRVDNLPNIKIATR |
| | KYLGKONVYDIGVER |
| SEQ ID No: 11 | MKGSSASIKIATRKYLGKQNVYDIGVERDH |
| | NFALKNGFIASNCFNGGENLYFQGRSSGSG |
| | SGGEYFTLQIRGRERFEEFREKNEALELKD |
| | AQAGKEPGGDSEVNQEAKPEVKPEVKPETH |
| | INLKVSDGSSEIFFKIKKTTPLRRLMEAFA |
| | KRQGKEMDSLRFLYDGIRIQADQTPEDLDM |
| | EDNDIIEAHREQIGGSGGSGGTCLSYETEI |
| | LTVEYGLLPIGKIVEKRIECTVYSVDNNGN |
| | IYTQPVAQWHDRGEQEVFEYCLEDGSLIRA |
| | TKDHKFMTVDGQMLPIDEIFERELDLMRVD |
| | NLPNVDSGSGETVRFQGGGSGGSSGDHNFA |
| | LKNGFIASNCFNGGHHHHHHELSGSGSGGE |
| | YFTLQIRGRERFEEFREKNEALELKDAQAG |
| | KEPGGSGGSGTSMSKGEELFTGVVPILVEL |
| | DGDVNGHKFSVRGEGEGDATNGKLTLKFIC |
| | TTGKLPVPWPTLVTTLTYGVQCFSRYPDHM |
| | KRHDFFKSAMPEGYVQERTISFKDDGTYKT |
| | RAEVKFEGDTLVNRIELKGIDFKEDGNILG |
| | HKLEYNFNSHNVYITADKQKNGIKANFKIR |
| | HNVEDGSVQLADHYQQNTPIGDGPVLLPDN |
| | HYLSTQSVLSKDPNEKRDHMVLLEFVTAAG |
| | ITHGMDELYKTSCLSYETEILTVEYGLLPI |
| | GKIVEKRIECTVYSVDNNGNIYTQPVAQWH |
| | DRGEQEVFEYCLEDGSLIRATKDHKFMTVD |
| | GQMLPIDEIFERELDLMRVDNLPNIKIATR |
| | KYLGKQNVYDIGVER |

### BRIEF DESCRIPTION OF THE DRAWINGS

FIG. 1 shows a schematic structural diagram of some of protein heterocatenanes synthesized by different orthogonal coupling reactions in the present disclosure, where L₁₋₁/L₁₋₂ and L₂₋₁/L₂₋₂ represent the two cyclization motifs in orthogonal modes; when the cyclization motifs are peptide-protein reactive pairs, side-chain coupling occurs and the resulting complexes are L₁ and L₂ respectively and will exist in the synthesized heterocatenanes; when the cyclization motifs are split inteins, the backbone coupling occurs and the resulting complexes are cleaved off the chain by self-splicing after the cyclization and will not exist in the synthesized heterocatenanes.

FIG. 2 shows two representative schematic diagram of protein heterocatenane syntheses by orthogonal coupling reactions in the present disclosure, in which (a) the biosynthesis of a protein heterocatenane is mediated by in situ protease digestion, SpyTag-SpyCatcher reactive pair and the split intein IntC1/IntN1; and (b) the biosynthesis of a protein heterocatenane is mediated by two orthogonal split inteins IntC1/IntN1 and IntC2/IntN2.

FIG. 3 shows the size exclusion chromatography of a protein heterocatenane cat-BXA-X synthesized in an example (a), the SDS-PAGE characterization results before and after TEV protease digestion (b), and the mass spectrum of cat-BXA-X (c).

FIG. 4 shows the size exclusion chromatography of a protein heterocatenane cat-(AffiHER2-BXA-AffiHER2)-X synthesized in an example (a), the SDS-PAGE characterization results before and after the TEV protease digestion (b), and the mass spectrum of cat-(AffiHER2-BXA-AffiHER2)-X (c).

FIG. 5 shows the size exclusion chromatography of a protein heterocatenane cat-XSUMO-X synthesized in an example (a), the SDS-PAGE characterization results before and after the TEV protease digestion (b), and the mass spectrum of cat-XSUMO-X (c).

FIG. 6 shows the size exclusion chromatography of a protein heterocatenane cat-XSUMO-XGFP synthesized in an example (a), the SDS-PAGE characterization results before and after the TEV protease digestion (b), and the mass spectrum of cat-XSUMO-XGFP (c).

FIG. 7 shows the mass spectra of the TEV protease digestion products of protein heterocatenanes synthesized in the examples, including I-BXA (a) and c-X (b) from the protein heterocatenane cat-BXA-X, I-AffiHER2-BXA-AffiHER2 (c) and c-X (d) from the protein heterocatenane cat-(AffiHER2-BXA-AffiHER2)-X, and I-XSUMO (e) and c-X (f) from the protein heterocatenane cat-XSUMO-X.

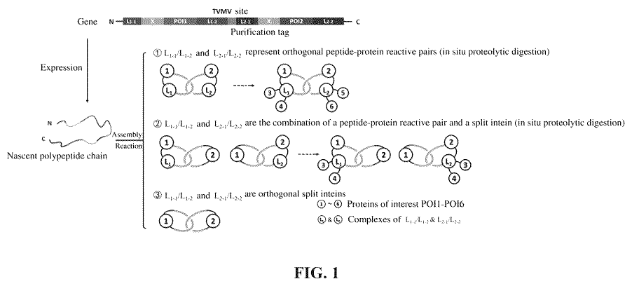

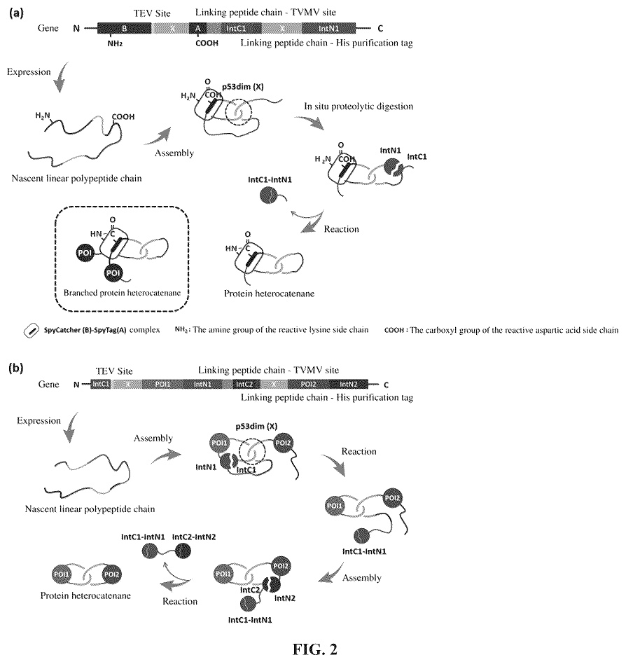

### DETAILED DESCRIPTION

The present disclosure is further described in detail below by way of examples, which are not intended to limit the scope of the present disclosure in any way.

Protein precursors involved in biosynthesis of protein heterocatenanes and their corresponding expression systems are constructed by the following specific steps:
1) For the system in which the synthesis of protein heterocatenanes is mediated jointly by the SpyTag-SpyCatcher reactive pair and the split intein IntC1/IntN1, a gene sequence containing a 6×His tag (for protein purification), SpyTag and SpyCatcher reactive pair, p53dim domains, a split intein IntC1/IntN1, i.e., SpyCatcher(B)-p53dim(X)-SpyTag(A)-IntC1-p53dim(X)-IntN1 (BXA-IntC1-X-IntN1) is constructed by the recombinant genetic engineering technique. On the basis of this gene sequence, a folded protein AffiHER2 is further introduced at the N terminus of the SpyCatcher and the C terminus of the SpyTag respectively to construct a gene sequence, i.e., AffiHER2-SpyCatcher(B)-p53dim(X)-SpyTag(A)-AffiHER2-IntC1-p53dim(X)-IntN1 (AffiHER2-BXA-AffiHER2-IntC1-X-IntN1). The two gene sequences are inserted into an expression vector pMSCG19 respectively, transformed into a pRK1037 plasmid-containing BL21(DE3) competent cell for expression. The pRK1037 plasmid can encode the TVMV protease. During the expression, the biosynthesis of protein heterocatenanes cat-BXA-X and cat-(AffiHER2-BXA-AffiHER2)-X is achieved by in situ assembly, protease digestion and site-specific cyclization.
2) For the system in which the synthesis of protein heterocatenanes is mediated by orthogonal split inteins, a gene sequence containing a 6-His tag (for protein purification), p53dim domains, split inteins IntC1/IntN1 and IntC2/IntN2, and a protein of interest SUMO/GFP, i.e., IntC1-p53dim-SUMO-IntN1-IntC2-p53dim-IntN2 (IntC1-X-SUMO-IntN1-IntC2-X-IntN2) or IntC1-p53dim-SUMO-IntN1-IntC2-p53dim-GFP-IntN2 (IntC1-X-SUMO-IntN1-IntC2-X-GFP-IntN2) is constructed by the recombinant genetic engineering technique. The two gene sequences are inserted into the expression vector pMSCG19 respectively, transformed into a BL21(DE3) competent cell for expression. During the expression, the biosynthesis of protein heterocatenanes cat-XSUMO-X and cat-XSUMO-XGFP is achieved by in situ assembly and the cyclization reactions mediated by orthogonal split inteins.

The prepared protein heterocatenanes are subjected to basic characterization and their topologies are proven through sodium dodecyl sulfate-polyacrylamide gel electrophoresis (SDS-PAGE), ultra-performance liquid chromatography-mass spectrometry (LC-MS), and TEV protease digestion reaction.

### Example 1: Biosynthesis of Protein Heterocatenanes Cat-BXA-X and cat-(AffiHER2-BXA-AffiHER2)-X using the co-expression system of pMCSG19/pRK1037

Gene fragments of BXA-IntC1-X-IntN1 and AffiHER2-BXA-AffiHER2-IntC1-X-IntN1 were inserted into the expression vectors pMCSG19 respectively, with the sequences shown in SEQ ID No:8 and SEQ ID No:9 in the sequence listing. The resulting constructs were confirmed by sequencing, then transformed into the pRK1037 plasmid-containing BL21(DE3) competent cells, and incubated overnight at 37° C. on Amp-Kan plates containing 100 μg/mL of sodium ampicillin (Amp) and 50 μg/mL of kanamycin (Kan). Thereafter, monoclonal colonies were picked out, inoculated into a 5-mL 2×YT medium with the same antibiotics, and subjected to shake incubation at 37° C. for 10 to 12 hours to prepare a seed broth. The seed broth was inoculated at a ratio of 1:100 into a 250 mL 2×YT medium with the same antibiotics, and the obtained cultures were subjected to shake incubation at 37° C. until OD₆₀₀ was between 0.5 and 0.7. Isopropyl-p-D-thiogalactopyranoside (IPTG) was added to a final concentration of 0.25 mM, and then the cultures were shaken at 16° C. for 20 hours for expression.

### Example 2: Biosynthesis of Protein Heterocatenanes Cat-XSUMO-X and Cat-XSUMO-XGFP

Gene fragments of IntC1-X-SUMO-IntN1-IntC2-X-IntN2 and IntC1-X-SUMO-IntN1-IntC2-X-GFP-IntN2 were inserted into the expression vectors pMCSG19 respectively, with the sequences shown in SEQ ID No:10 and SEQ ID No:11 in the sequence listing. The resulting constructs were confirmed by sequencing, then transformed into BL21(DE3) competent cells, and incubated overnight at 37° C. on plates containing 100 μg/mL of sodium ampicillin. Thereafter, monoclonal colonies were picked out, inoculated into a 5-mL 2-YT medium with the same antibiotics, and subjected to shake incubation at 37° C. for 10 to 12 hours to prepare a seed broth. The seed broth was inoculated at a ratio of 1:100 into a 250 mL 2×YT medium with the same antibiotics, and the obtained cultures were subjected to shake incubation at 37° C. until OD₆₀₀ was between 0.5 and 0.7. Isopropyl-β-D-thiogalactopyranoside (IPTG) was added to a final concentration of 0.25 mM, and then the cultures were shaken at 16° C. for 20 hours for expression.

### Example 3: Purification of Protein Heterocatenanes

Upon completion of the protein expression, the bacterial cells were collected by centrifugation (5500 g×15 min) with a high-speed refrigerated centrifuge and the supernatant was discarded. Bacterial cells were re-suspended with lysis buffer A (50 mM sodium dihydrogen phosphate, 300 mM sodium chloride, 10 mM imidazole, pH 8.0). The re-suspension was sonicated with an ultrasonic homogenizer in an ice-water bath (5-second interval for every 5-second operation, 30% intensity) and then centrifuged (12000 g×30 min) to collect the supernatant. The supernatant was mixed well with Ni-NTA resin and incubated at 4° C. for 1 hour. The mixture was poured into an empty column PD-10 for purification, and after the lysate was exhausted, the resin was washed with wash buffer B (50 mM sodium dihydrogen phosphate, 300 mM sodium chloride, 20 mM imidazole, pH 8.0) for 5 to 10 times the resin volume to reduce non-specific adsorption. The protein heterocatenanes cat-BXA-X, cat-(AffiHER2-BXA-AffiHER2)-X and cat-XSUMO-X could be eluted directly with elution buffer C (50 mM sodium dihydrogen phosphate, 300 mM sodium chloride, 250 mM imidazole, pH 8.0). In order to increase the purity, the protein heterocatenane cat-XSUMO-XGFP was subjected to gradient elution of first eluting with elution buffer D (50 mM sodium dihydrogen phosphate, 300 mM sodium chloride, 50 mM imidazole, pH 8.0) for about 10 times the resin volume, collecting the protein eluent which mainly contained heterocatenane, and then eluting the cyclic or catenated by-product of GFP with the elution buffer C.

The protein eluent was further purified using a fast protein liquid chromatography system (ÄKTA pure, GE Healthcare) with a size exclusion chromatography column (Superdex 200 increase 10/300 GL, GE Healthcare). The mobile phase was phosphate buffered saline PBS (pH 7.4) filtered through a 0.22 μm filter at a flow rate of 0.5 mL % min. The efflux peak of the protein was monitored by UV absorption at 280 nm, and the sample was collected for characterization.

### Example 4: Characterization of Protein Heterocatenanes

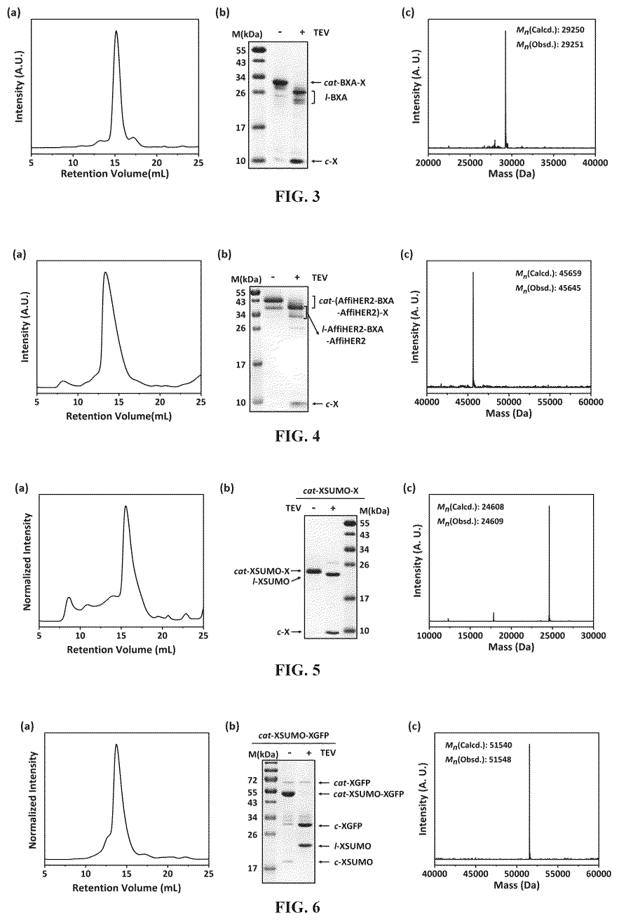

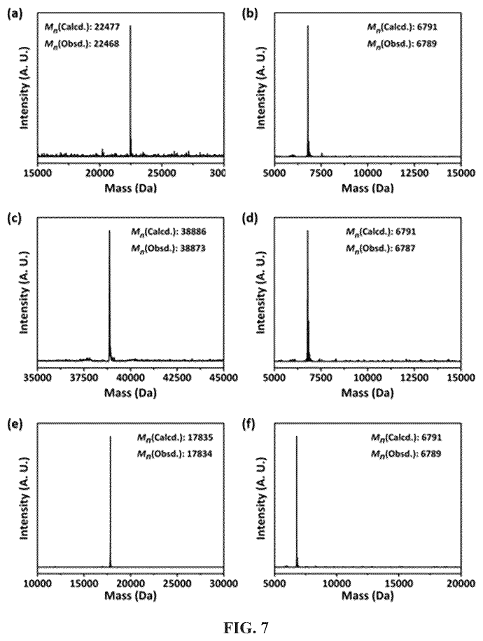

The protein heterocatenanes purified in Example 3 were first added with 5×SDS loading buffer, heated at 98° C. for 10 min, and then characterized by SDS-PAGE. After exchanging buffers of the protein samples purified by SEC into ddH₂O with an ultrafiltration tube, LC-MS was adopted to characterize their molecular weights. Protein concentrations were determined by an ultra-micro spectrophotometer (NanoPhotometer P330, Implen, Inc.). To prove the heterocatenane topology, the protein solution (10 μM) and TEV protease solution (10 μM) were mixed at a molar ratio of 20:1 and proteolysis was carried out at 37° C. (for 1, 3, 6 hours, where the protease digestion could be substantially complete within 3 hours). After the protease digestion, 10 μL of the proteolytic products were added with 5×SDS loading buffer and heated at 98° C. for 10 min to quench the reaction. The product composition after digestion was characterized by SDS-PAGE. After exchanging buffers of the remaining digested system into ddH₂O with an ultrafiltration tube, LC-MS was employed to confirm the molecular weight of the proteolytic products. The results of the SEC characterization after affinity purification by a nickel column, SDS-PAGE characterization before and after the enzyme digestion, and the LC-MS characterization of the cat-BXA-X, cat-(AffiHER2-BXA-AffiHER2)-X, cat-XSUMO-X, and cat-XSUMO-XGFP were as shown in FIGS. 3, 4, 5 and 6, respectively. The LC-MS characterizations of the proteolytic products of the cat-BXA-X, cat-(AffiHER2-BXA-AffiHER2)-X, and cat-XSUMO-X by TEV protease digestion were as shown in FIG. 7.

What is claimed is:

1. A method for biosynthesis of a protein heterocatenane, comprising the following steps:
1) designing a protein precursor sequence of the protein heterocatenane with a basic structure including, from N terminus to C terminus: L₁₋₁-X-L₁₋₂-(in situ protease digestion site)-L₂₋₁-X-L₂₋₂, wherein X represents a dimer-forming entwining motif, which may be homodimeric or heterodimeric, that is, two Xs may or may not be the same; L₁₋₁/L₁₋₂ and L₂₋₁/L₂₋₂ represent two pairs of cyclization motifs that undergo an orthogonal coupling reaction in cellulo, and the two pairs of cyclization motifs may be two orthogonal peptide-protein reactive pairs, or combinations of a peptide-protein reactive pair and a split intein, or two orthogonal split inteins; when L₁₋₁/L₁₋₂ is the peptide-protein reactive pair, the in situ protease digestion site inserted between L₁₋₂ and L₂₋₁ is an essential element, which can be digested in situ by co-expressing a protease intracellularly; otherwise the in situ protease digestion site is a non-essential element; the sequence of a protein of interest is inserted in the basic structure with the insertion sites selected from: before and/or after an X domain, at the N terminus and/or at the C terminus of the peptide-protein reactive pair;
2) constructing a gene sequence encoding the corresponding protein precursor sequence according to step 1) and introducing the gene sequence into an expression vector;
3) transforming the expression vector constructed in step 2) into a cell for expression, and co-expressing, if necessary, the protease that in situ cleaves the digestion site in cellulo; and
4) purifying a fusion protein obtained in step 3) to obtain a corresponding protein heterocatenane.

2. The method according to claim 1, wherein the entwining motif in step 1) is a p53dim domain or a p53dim mutant capable of forming a dimeric structure, where the amino acid sequence of the p53dim domain is as shown in SEQ ID NO:3 in the sequence listing.

3. The method according to claim 1, wherein the peptide-protein reactive pair in step 1) is selected from a SpyTag-SpyCatcher reactive pair and a SnoopTag-SnoopCatcher reactive pair.

4. The method according to claim 3, wherein the amino acid sequences of SpyTag and SpyCatcher in the SpyTag-SpyCatcher reactive pair are as shown in SEQ ID NO:1 and SEQ ID NO:2 in the sequence listing, respectively.

5. The method according to claim 1, wherein the split intein in step 1) is an NpuDnaE split intein, which consists of IntC1 and IntN1 or IntC2 and IntN2 as a cyclization motif, and the amino acid sequences of IntC1, IntN1, IntC2, and IntN2 are as shown in SEQ ID NO:4, SEQ ID NO:5, SEQ ID NO:6, and SEQ ID NO:7 in the sequence listing, respectively.

6. The method according to claim 1, wherein the in situ protease digestion site designed in step 1) is a recognition sequence ETVRFQG of a TVMV protease or a recognition sequence ENLYFQG of a TEV protease; and accordingly the TVMV protease or the TEV protease is co-expressed in step 3).

7. The method according to claim 1, wherein a histidine tag sequence is introduced before a second entwining motif X in step 1), and protein purification is performed by affinity chromatography on a nickel column in step 4).

8. The method according to claim 1, wherein the basic structure of the protein precursor sequence designed in step 1) is SpyCatcher-p53dim-SpyTag-IntC1-p53dim-IntN1, which is, from N terminus to C terminus in order, a cyclization reaction motif SpyCatcher, an entwining motif p53dim domain, a cyclization reaction motif SpyTag, a C-terminal part IntC1 of the split intein, an entwining motif p53dim domain, and an N-terminal part IntN1 of the split intein; a recognition sequence of a TVMV protease is inserted between SpyTag and IntC1, and a histidine tag sequence is introduced before a second p53dim domain; a fusion site for one or more identical or different proteins of interest is selected from: before and/or after the p53dim domain, at the N terminus of SpyCatcher, and at the C terminus of SpyTag.

9. The method according to claim 1, wherein the basic structure of the protein precursor sequence designed in step 1) is IntC1-p53dim-IntN1-IntC2-p53dim-IntN2, which is, from the N terminus to the C terminus in order, a C-terminal part IntC1 of the split intein, an entwining motif p53dim domain, an N-terminal part IntN1 of the split intein, a C-terminal part IntC2 of the split intein, an entwining motif p53dim domain, and an N-terminal part IntN2 of the split intein; a histidine tag sequence is introduced before a second p53dim domain; and one or more identical or different proteins of interest are inserted before and/or after two p53dim domains.

10. The method according to claim 1, wherein for the protein heterocatenane in which a histidine tag sequence is introduced in step 4), an expressed protein is purified by affinity chromatography on a nickel column, and the purity of the protein heterocatenane is further improved by gradient elution or size exclusion chromatography.


SEQUENCE LISTING (11 sequences)

SEQ ID NO: 1 - protein, 13 aa, Artificial Sequence
  Synthesized
AHIVMVDAYKPTK

SEQ ID NO: 2 - protein, 115 aa, Artificial Sequence
  Synthesized
AMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 3 - protein, 38 aa, Artificial Sequence
  Synthesized
GGEYFTLQIRGRERFEEFREKNEALELKDAQAGKEPGG

SEQ ID NO: 4 - protein, 35 aa, Artificial Sequence
  Synthesized
IKIATRKYLGKQNVYDIGVERDHNFALKNGFIASN

SEQ ID NO: 5 - protein, 102 aa, Artificial Sequence
  Synthesized
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 6 - protein, 14 aa, Artificial Sequence
  Synthesized
DHNFALKNGFIASN

SEQ ID NO: 7 - protein, 123 aa, Artificial Sequence
  Synthesized
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVER

SEQ ID NO: 8 - protein, 420 aa, Artificial Sequence
  Synthesized
MKGSSASAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQENLYFQGGSGSGGEYFTLQIRGRERFEEFREKNEALELKDAQAGKEPGGSGGSGAHIVMVDAYKPTKVDSGSGETVRFQGGGSGGSSGMIKIATRKYLGKQNVYDIGVERDHNFALKNGFIASNCFNGGHHHHHHELSGSGSGGEYFTLQIRGRERFEEFREKNEALELKDAQAGKEPGGSGGSGTSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 9 - protein, 570 aa, Artificial Sequence
  Synthesized
MKGSSTGGQQMGRDPGVDNKFNKEMRNAYWEIALLPNLNNQQKRAFIRSLYDDPSQSANLLAEAKKLNDAQAPKGGGGSASAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQENLYFQGGSGSGGEYFTLQIRGRERFEEFREKNEALELKDAQAGKEPGGSGGSGAHIVMVDAYKPTKGTGGSMTGGQQMGRDPGVDNKFNKEMRNAYWEIALLPNLNNQQKRAFIRSLYDDPSQSANLLAEAKKLNDAQAPKGVDSGSGETVRFQGGGSGGSSGMIKIATRKYLGKQNVYDIGVERDHNFALKNGFIASNCFNGGHHHHHHELSGSGSGGEYFTLQIRGRERFEEFREKNEALELKDAQAGKEPGGSGGSGTSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 10 - protein, 525 aa, Artificial Sequence
  Synthesized
MKGSSASIKIATRKYLGKQNVYDIGVERDHNFALKNGFIASNCFNGGENLYFQGRSSGSGSGGEYFTLQIRGRERFEEFREKNEALELKDAQAGKEPGGDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSGGSGGTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNVDSGSGETVRFQGGGSGGSSGDHNFALKNGFIASNCFNGGHHHHHHELSGSGSGGEYFTLQIRGRERFEEFREKNEALELKDAQAGKEPGGSGGSGTSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVER

SEQ ID NO: 11 - protein, 765 aa, Artificial Sequence
  Synthesized
MKGSSASIKIATRKYLGKQNVYDIGVERDHNFALKNGFIASNCFNGGENLYFQGRSSGSGSGGEYFTLQIRGRERFEEFREKNEALELKDAQAGKEPGGDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSGGSGGTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNVDSGSGETVRFQGGGSGGSSGDHNFALKNGFIASNCFNGGHHHHHHELSGSGSGGEYFTLQIRGRERFEEFREKNEALELKDAQAGKEPGGSGGSGTSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKTSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVER